(19) United States
(12) Patent Application Publication
(10) Pub. No.: US 2025/0387542 A1
(43) Pub. Date: Dec. 25, 2025

(54) SWELLABLE HYDROGEL GRAFT FOR EXUDATE CONTROL

(71) Applicant: Tides Medical LLC, Lafayette, LA (US)

(72) Inventors: Mohammadali Safavieh, North Andover, MA (US); Mora Carolyn Melican, Weston, MA (US)

(73) Assignee: Tides Medical LLC, Lafayette, LA (US)

(21) Appl. No.: 18/752,746

(22) Filed: Jun. 24, 2024

(51) Int. Cl. A61L 27/52

(52) U.S. Cl. CPC A61L 27/52; A61L 2300/60

(57) ABSTRACT

This disclosure generally relates to grafts having exudate control for treating a wound of a subject and methods thereof. In some embodiments, the grafts comprising a first layer comprising a first portion and a second portion. In some embodiments, the first portion comprises amnion, chorion, or a combination thereof. In some embodiments, the second portion comprises a swellable hydrogel. In other embodiments, the grafts comprise a first layer comprising amnion, chorion, or a combination thereof and a second layer, disposed on the first layer, comprising the swellable hydrogel. Additionally, in some embodiments, the grafts may further comprise one or more additional layers, for example, a third layer, adjacent the second layer, comprising an adsorbent material; and/or an adhesive fourth layer adjacent the third layer for adhering said graft to the skin of a subject. Other embodiments are directed toward methods of bonding two or more layers within the graft.

### FIELD

The present disclosure generally relates to grafts having exudate control for treating a wound of a subject.

### BACKGROUND

Allografts derived from placental tissue, such as amnion grafts, chorion grafts, and amnion/chorion combination grafts, are commonly used as wound dressings, for example, to treat non-healing chronic wounds (e.g., wounds that fail to proceed through the normal phases of wound healing in an orderly and timely manner). One of the major challenges associated with using placental tissues as wound dressings is that current products do not enable management of wound exudate. Even though a moist wound environment is necessary for optimal healing, over- or under-production of exudate may adversely affect healing. It is known in the art that chronic wounds are associated with higher levels of exudate production, which may impede healing as it not only makes it hard to control infection and biofilm formation, but also slows down or even prevents cell proliferation, interferes with growth factor availability and contains elevated levels of inflammatory mediators and proteases. Accordingly, improved solutions and methods are needed.

### SUMMARY

The subject matter of the present disclosure involves, in some cases, interrelated products, alternative solutions to a particular problem, and/or a plurality of different uses of one or more systems and/or articles.

Aspects of the present disclosure are generally directed toward grafts having exudate control for treating a wound of a subject. In some embodiments, the graft comprising a first layer comprising a first portion comprising amnion, chorion, or a combination thereof; and a second portion adjacent the first portion, comprising a swellable hydrogel. In some embodiments the swellable hydrogel comprises a hydrophilic polymer and a polysaccharide.

Other embodiments are directed toward a graft comprising a first layer comprising amnion, chorion, or a combination thereof; and a second layer, disposed on the first layer, comprising a swellable hydrogel. In some embodiments, the swellable hydrogel comprises a hydrophilic polymer and a polysaccharide.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In another aspect, the present disclosure encompasses methods of making one or more of the embodiments described herein, for example, a graft having exudate control. In still another aspect, the present disclosure encompasses methods of using one or more of the embodiments described herein, for example, grafts having exudate control.

Other advantages and novel features of the present disclosure will become apparent from the following detailed description of various non-limiting embodiments of the disclosure when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A and 1B show a top and cross-sectional view, respectively, of an exemplary graft comprising a first layer comprising a first portion and a second portion, adjacent to and embedded within the first portion. FIG. 1B further illustrates one possible method for securing the second portion within a hole of the first portion via a lip. In some embodiments, an adhesive may be applied to the lip and used to bond the second layer within a hole of the first layer.

FIG. 2 shows a cross-sectional view of an exemplary graft comprising a second layer adjacent to a first layer as described in FIG. 1. In some embodiments, the second layer is a (super) absorbent material. FIG. 2 also shows a third layer adjacent to the second layer. In some embodiments, the third layer comprises an adhesive material.

FIGS. 3A and 3B show a top and cross-sectional view, respectively, of an exemplary first layer comprising amnion, chorion, and/or a combination thereof, comprising a hole.

FIGS. 4A and 4B show a top and cross-sectional view, respectively, of an exemplary first layer comprising amnion, chorion, and/or a combination thereof, comprising a plurality of holes.

FIGS. 5 and 6 shows a top and cross-sectional view, respectively of an exemplary graft comprising a first layer comprising amnion, chorion, or a combination thereof, divided into a plurality of segments, wherein each segment is disposed directly onto a first side of a second layer comprising a swellable hydrogel.

FIG. 7 shows a cross-sectional view of an exemplary graft comprising a second layer comprising a swellable hydrogel disposed on a first layer comprising amnion, chorion, and/or a combination thereof. FIG. 7 also shows a third layer comprising a (super) absorbent material disposed on the second layer, and a fourth layer comprising an adhesive material disposed on the third layer.

FIG. 8 shows the gravimetric swelling of hydrogels, contemplated herein, comprising 10% wt of Guar Gum in a solution of phosphate buffered saline in a pH range of between 5-6 from 0 hours to 72 hours.

FIG. 9 shows the gravimetric swelling of hydrogels comprising guar gum (10 wt %) and polyethylene glycol diacrylate 20K (PEGDA, 15 wt %) in a PBS solution at pH 5.5, 7.5 or 8.5 from 0 hours to 336 hours.

FIG. 10 shows the gravimetric swelling of hydrogels comprising PEGDA only (15 wt %) or a combination of PEGDA (15 wt %) and Guar Gum (10 wt %). Hydrogels comprising both the hydrophilic polymer and the saccharide exhibited lower swelling ratios than hydrogels comprising the hydrophilic polymer alone.

FIGS. 11A and 11B show exemplary grafts comprising a second layer comprising a swellable hydrogel and a third layer comprising a (super) absorbent material, wherein the layers were attached using cyanoacrylate glue. In FIG. 11A, the second layer and third layer were attached by applying the cyanoacrylate glue to the outer edge of the third layer. In FIG. 11B, the second layer and third layer were attached by applying a cyanoacrylate dot in the center of the third layer. As shown in FIG. 11B, the cyanoacrylate dots allowed the hydrogel to swell freely and did not lead to warping as observed in FIG. 11A.

FIGS. 12A and 12B show exemplary grafts comprising a second layer comprising a swellable hydrogel and a third layer comprising a (super) absorbent material, wherein the swellable hydrogel is crosslinked, at least partially, around the (super) absorbent material. This is accomplished by infusing a swellable hydrogel liquid photopolymerizable precursor solution directly into the (super) absorbent material and subsequently photopolymerizing the swellable hydrogel, within at least a part, of the (super) absorbent material.

FIGS. 13A and 13B show exemplary grafts described in FIG. 12 immediately after addition to a liquid solution (FIG. 13A) and after achieving equilibrium swelling (FIG. 13B).

FIGS. 14A and 14B show exemplary grafts described in FIG. 11 immediately after addition to a liquid solution (FIG. 14A) and after achieving equilibrium swelling (FIG. 14B).

FIG. 15A shows an exemplary first layer comprising amnion, chorion, or a combination thereof, divided into a plurality of segments. FIG. 15B shows an exemplary graft wherein each segment is disposed directly onto a first side of a second layer comprising a swellable hydrogel.

FIG. 16 shows one example of a fully fabricated exudate control graft with integrated Human dried placenta graft of Artacent membrane as contemplated herein.

### DETAILED DESCRIPTION

This disclosure generally relates to grafts having exudate control for treating a wound of a subject and methods thereof. In some embodiments, the grafts comprising a first layer comprising a first portion and a second portion. In some embodiments, the first portion comprises amnion, chorion, Wharton's jelly, cord, or a combination thereof. In some embodiments, the second portion comprises a swellable hydrogel. In other embodiments, the grafts comprise a first layer comprising amnion, chorion, Wharton's jelly, cord, or a combination thereof and a second layer, disposed on the first layer, comprising the swellable hydrogel. Additionally, in some embodiments, the grafts may further comprise one or more additional layers, for example, a third layer, adjacent the second layer, comprising an adsorbent material; and/or an adhesive fourth layer adjacent the third layer for adhering said graft to the skin of a subject. Other embodiments are directed toward methods of bonding two or more layers within the graft.

Grafts derived from placental tissue, such as amnion grafts, chorion grafts, and amnion/chorion combination grafts, are commonly used as wound dressings, for example, to treat non-healing chronic wounds. A major challenge associated with these grafts, however, is the management of exudate generated in the wound bed during healing. While it is generally accepted in the art that wound exudates produced during the early stages of wound healing are beneficial for repair, excessive exudates can overhydrate the wound, inhibit fibroblast production, increase risk of infection, and prolong the inflammatory phase, thereby impeding the healing process.

Accordingly, one aspect of the present disclosure is directed to the discovery that grafts comprising a swellable hydrogel can regulate the wound exudate over various timescales. Without wishing to be bound by any particular theory, it is generally believed that by placing a swellable hydrogel in contact with the exudate, excessive exudate will be wicked into the hydrogel and away from the wound bed. Those of skill in the art will understand that the swellability of the hydrogel and the rate at which the exudate is wicked away can be tailored by manipulating various materials properties of the hydrogel (e.g., chemical composition, crosslinking density, etc.).

It has also been discovered that placement of a (super) absorbent material adjacent to the swellable hydrogel increases the volume of exudate retained by a graft. This is important because it reduces the risk of wound infection associated with repeated replacement of soiled wound dressings. As such, in some embodiments, the grafts disclosed here comprise a (super) absorbent material, such as, for example, Gelfoam®.

In addition to exudate control, the skilled artisan will understand that the swellable hydrogel and/or a (super) absorbent material may be loaded with one or more therapeutics, for example, to aid in wound healing or to prevent infection. In some embodiments, the one or more therapeutics may be released into the wound bed upon swelling of the hydrogel and/or (super) absorbent material; however, in some embodiments, the therapeutic is too large to be released from the hydrogel network (e.g., it remains entrapped within the hydrogel even after swelling, e.g., an antimicrobial nanoparticle). In other embodiments, the therapeutic is release from the hydrogel network and/or (super) absorbent material after swelling (e.g., antimicrobial, antifungal, small peptides, etc.).

The grafts of the present disclosure may be assembled in any suitable configuration using any suitable method known to the skilled artisan. For example, in some embodiments, a layer comprises a first portion comprising amnion, chorion, Wharton's jelly, cord, and/or a combination thereof, and a second portion comprising a swellable hydrogel. In some embodiments, the second portion is embedded within a hole of the first portion. Such configurations place the swellable hydrogel (e.g., second portion) in direct contact with the exudate in the wound bed. Other configurations are possible in other embodiments. For example, in some embodiments, a second layer, comprising the swellable hydrogel, is disposed on top the first layer comprising amnion, chorion, Wharton's jelly, cord, or a combination thereof, wherein the first layer further comprises either a single large hole or a plurality of smaller holes. In such configurations, the exudate must pass through the one or more holes of the first layer to reach the second layer comprising the swellable hydrogel.

Other configurations are also contemplated herein. For example, in some embodiments a first layer comprising amnion, chorion, Wharton's jelly, cord, or a combination thereof, is divided into a plurality of segments and each segment is disposed directly onto a first side of a second layer comprising a swellable hydrogel. In some embodiments, a first segment and second segment are separated by a first gap along a first axis and a second gap along a second axis (e.g., a grid-like pattern). Such configurations allow the exudate to flow through the gap between the plurality of segments of the first layer and into the second layer comprising the swellable hydrogel. Without wishing to be bound by any particular theory, it is believed that such configurations reduce swelling-induced delamination of the graft layers following absorption of the exudate by the swellable hydrogel.

In some embodiments, the first portion is in the form of a membrane. In some embodiments, the first portion is in the form of suspended particles (e.g., suspended particles comprising amnion, chorion, Wharton's jelly, and/or cord). In some embodiments, the first portion comprises a layer formed by 3D printing (e.g., additive manufacturing, layer by layer, or the like). In some embodiments, the first portion comprises fibers (e.g., fibers comprising amnion, chorion, Wharton's jelly, cord, or combinations thereof). In some such embodiments, the fibers may be spun into a mat. In some embodiments, the fibers may be bundled. In some embodiments, the fibers may be formed into a mesh. In some embodiments, at least a portion of the first portion has been lyophilized into a foam (e.g., the first portion comprises amnion, chorion, Wharton's jelly, and/or cord lyophilized into a foam). Other configurations and combinations (e.g., fibers and foam, particles and fibers) are also possible.

The following is a description of various layers (e.g., a first layer, second layer, etc.) and configurations of said layer(s) as contemplated herein, as well as exemplary grafts comprising said layer(s).

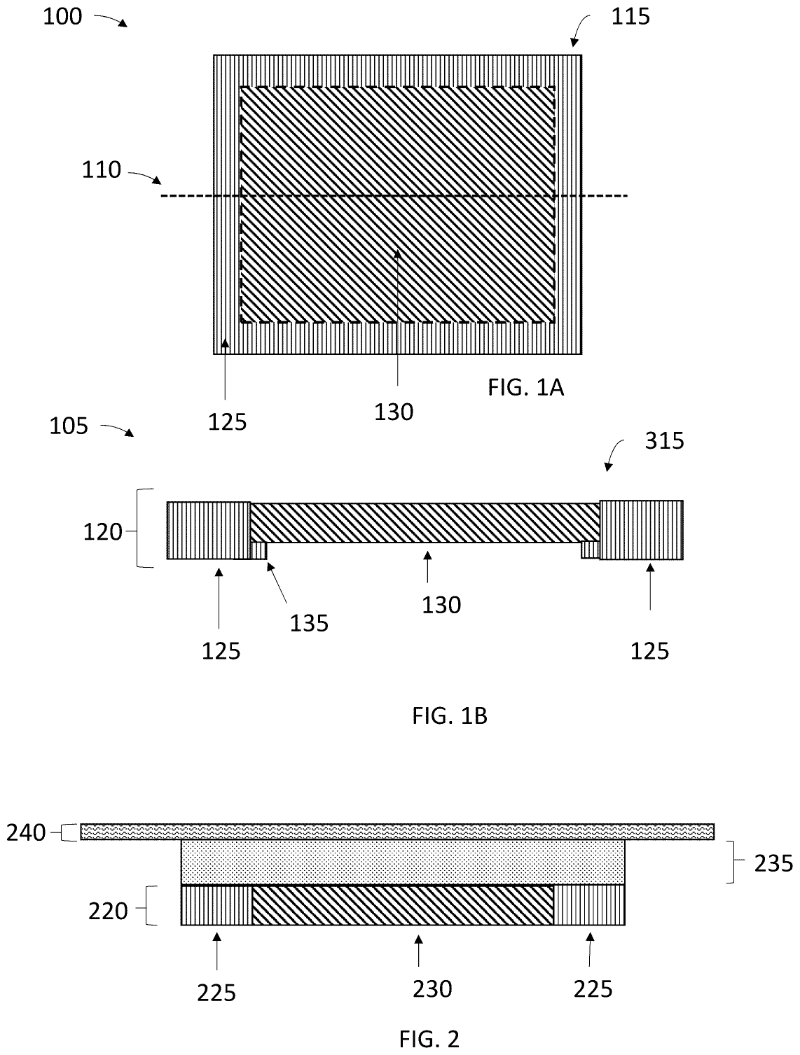

In some embodiments, a graft comprises a first layer comprising a first portion and a second portion. FIG. 1A shows a top view 100 and FIG. 1B shows cross-sectional view 105 (along axis 110) of an exemplary graft 115 comprising first layer 120. In some embodiments, first layer 120 comprises first portion 125 comprising amnion, chorion, Wharton's jelly, cord, and/or a combination thereof, and second portion 130, adjacent to first portion 125, comprising a swellable hydrogel. In some embodiments, second portion 130 is disposed within a hole (border of hole is shown as dotted line around second portion 130 in FIG. 1A) of first portion 125. In some embodiments, the swellable hydrogel is in a dried state (e.g., dehydrated). In some embodiments, second portion 130 is secured to lip 135 of first portion 125, for example, using cyanoacrylate glue (or any other suitable method known to one of skill in the art). However, it should be noted that while FIG. 1 shows lip 135 for securing second portion 130 to first portion 125, any suitable configuration and/or method known to the skilled artisan may be used to secure second portion 130 to first portion 125.

In some embodiments, a graft further comprises a second layer adjacent the first layer. For example, FIG. 2 shows a cross-sectional view of exemplary graft 200 comprising second layer 235 adjacent first layer 220. In some embodiments, first layer 220 comprises first portion 225 comprising amnion, chorion, Wharton's jelly, cord, and/or a combination thereof and second portion 230, adjacent the first portion, comprising a swellable hydrogel. In some embodiments, the second layer comprises a (super) absorbent material. In some embodiments, the (super) absorbent material comprises a hydrophilic material. Again, without being bound by any particular theory, it is believed that placement of a (super) absorbent material adjacent a swellable hydrogel acts as a “sink” that continually drives exudate from the wound bed into the (super) absorbent material via the swellable hydrogel.

In some embodiments, a graft further comprises a third layer adjacent the second layer. For example, FIG. 2 shows third layer 240 adjacent to second layer 235. In some embodiments, the third layer comprises an adhesive, for example, for securing the graft to a subject in need. Any suitable adhesive known to the skilled artisan may be used herein (e.g., cyanoacrylate). In some embodiments, the third layer is Tegaderm®.

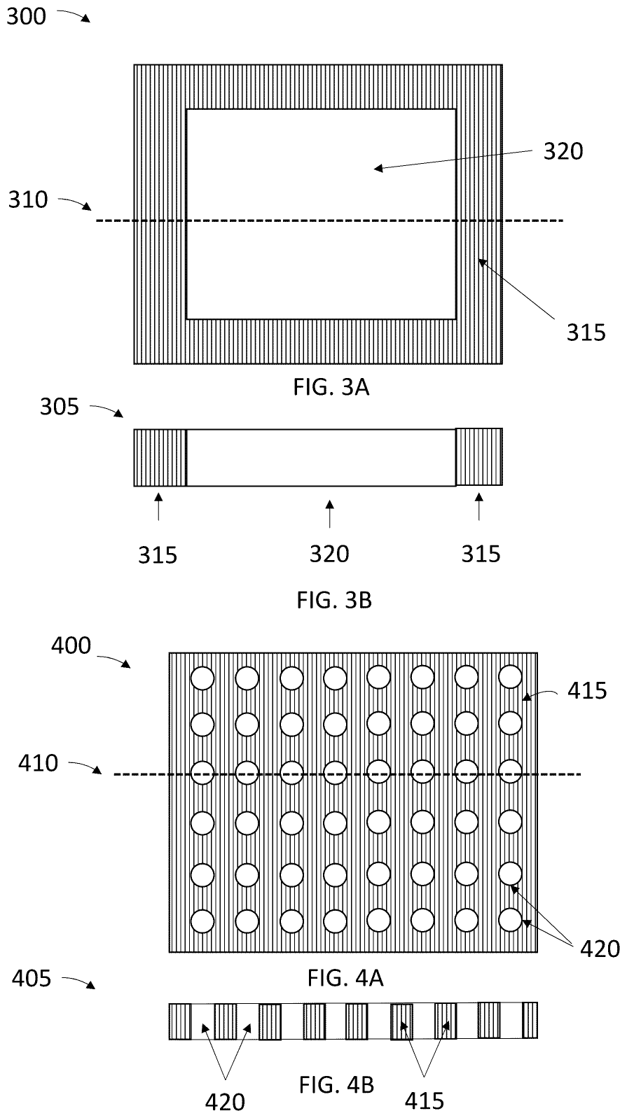

In some embodiments, a graft comprises a first layer and a second layer. In some embodiments, the first layer comprises amnion, chorion, Wharton's jelly, cord, or a combination thereof. In some embodiments, the second layer comprises a swellable hydrogel. In some embodiments, the first layer comprises a single hole. Alternatively, in some embodiments, the first layer comprises a plurality of holes. For example, FIG. 3A shows top view 300 and FIG. 3B shows cross-sectional view 305 (along axis 310) of exemplary first layer 315 with single hole 320; and FIG. 4A shows top view 400 and FIG. 4B shows cross-sectional view 405 (along axis 410) of exemplary first layer 415 having a plurality of holes 420. It is noted, that while FIG. 3 shows the hole centrally located within the first layer, the hole may be placed in any location within the first layer. Similarly, while FIG. 4 illustrates the plurality of holes as having a grid-like pattern, the plurality of holes may be arranged in any suitable manner known to the skilled artisan.

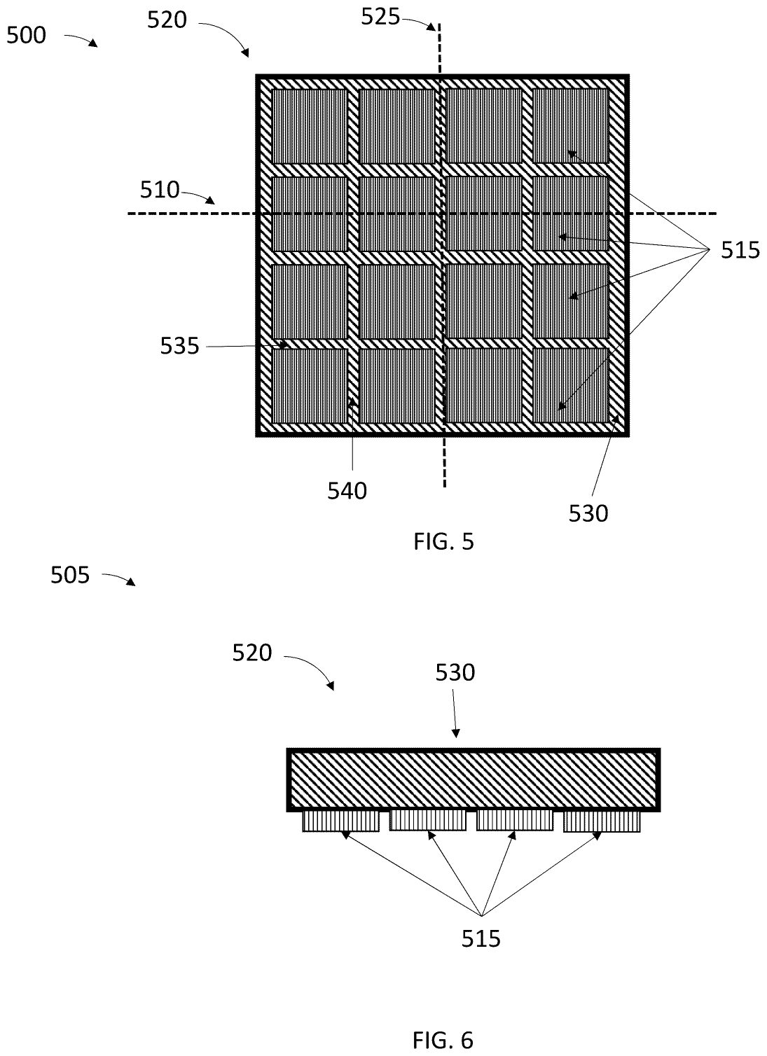

In some embodiments, a graft comprises a first layer comprising amnion, chorion, Wharton's jelly, cord, or a combination thereof, divided into a plurality of segments, wherein each segment is disposed directly onto a first side of a second layer comprising a swellable hydrogel. For example, FIG. 5 and FIG. 6 show a top view 500 and cross-sectional view 505 (along axis 510), respectively, of exemplary graft 520 comprising a first layer divided into plurality of segments 615. In some embodiments, a first segment and second segment are separated by gap 535 along first axis 525 and second gap 540 along a second axis 510 (e.g., a grid-like pattern). Such configurations allow the exudate to flow through the gaps (e.g., between adjacent segments) of the first layer and into the second layer comprising the swellable hydrogel.

It is to be noted that while FIGS. 5 and 6 show the plurality of segments as squares, the skilled artisan will understand that the segment (e.g., amnion and/or chorion) may be formed into any geometric shape known in the art, such as for example, regular geometric shapes and/or irregular geometric shapes (e.g., irregular circles). As used herein, the term “irregular geometric shape” generally refers to a any regular geometric shape with at least one distorted angle, point, and/or line. Exemplary geometric shapes include for example, squares, circles, rectangles, triangles, polygons, parallelogram, irregular squares, irregular circles, irregular rectangles, irregular triangles, irregular polygons, and/or irregular parallelograms. Other shapes are also contemplated in other embodiments.

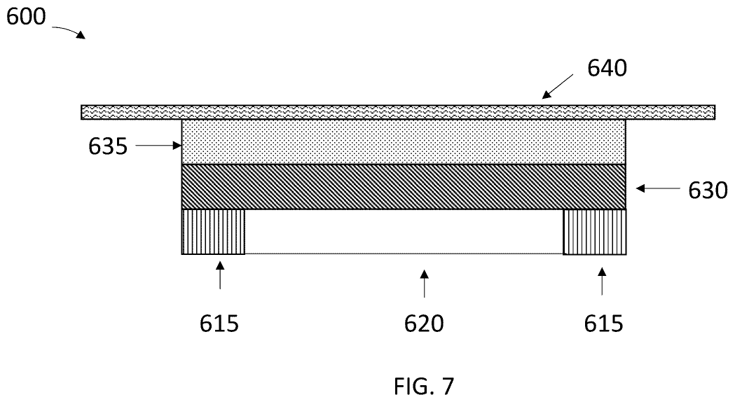

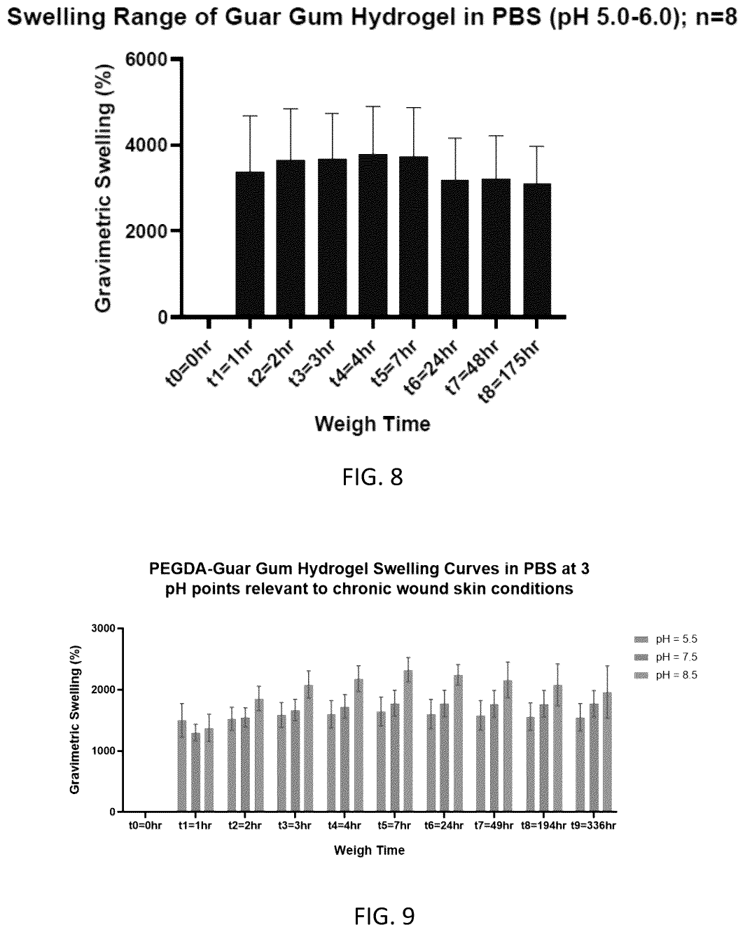

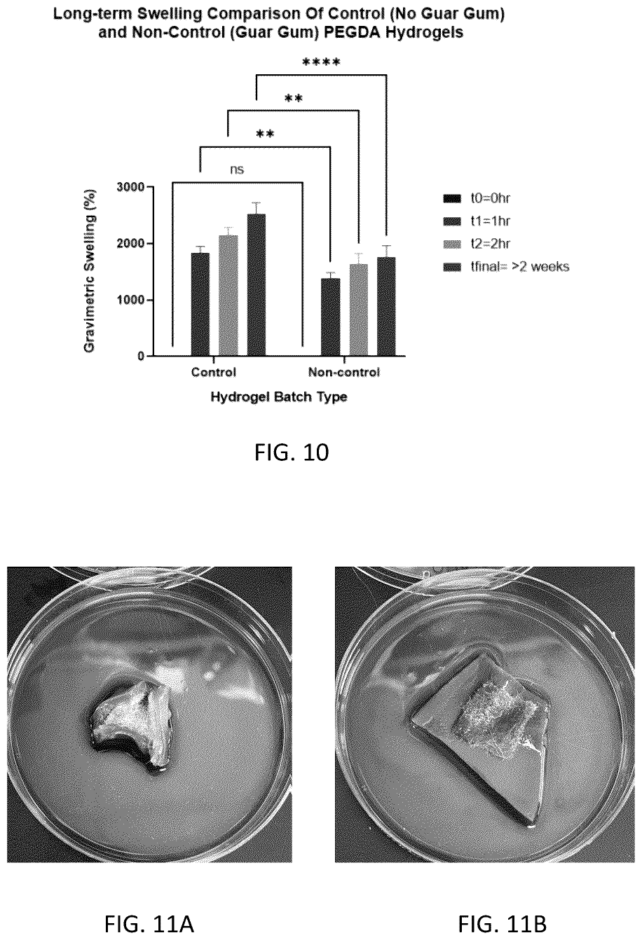

In some embodiments, a graft comprises one or more additional layers (e.g., a third layer, a fourth layer, etc.). For example, FIG. 7 shows a cross-sectional view of exemplary graft 600 comprising first layer 615 comprising amnion, chorion, Wharton's jelly, cord, and/or a combination thereof, with single hole 620, second layer 630 comprising a swellable hydrogel, disposed on first layer 615, third layer 635 comprising a (super) absorbent material disposed on second layer 630, and fourth layer 640 comprising an adhesive disposed on third layer 635.

In some embodiments, a swellable hydrogel, as disclosed herein, comprises a hydrophilic polymer and a polysaccharide. Any suitable hydrophilic polymer and/or any suitable polysaccharide may be used to form the swellable hydrogels disclosed herein. In some embodiments, the hydrophilic polymer comprises polyethylene glycol, polyethylene glycol acrylate, polyethylene glycol diacrylate, polyethylene glycol methacrylate, polyacrylamide, polyvinyl alcohol, polyoxaester, Pluronic, dimethylaminoethyl methacrylate, poly(2-hydroxyethyl methacrylate), hyaluronic acid, poly (N-isopropylacrylamide), poly ethylene oxide, poly oxaesters, chitosan, and dextran, or dimethylaminoethyl methacrylate. In some embodiments, the polysaccharide comprises Guar Gum, Arabic Gum, Gellan Gum, Xanthan Gum, agar, pectin, starch, carrageenan, pectin and/or alginate.

In some embodiments, a hydrophilic polymer within the swellable hydrogel has a concentration of between 0.1% (wt/wt) to about 20% (wt/wt). In some embodiments, the hydrophilic polymer is present within the swellable hydrogel at a concentration greater than or equal to 0.1% (wt/wt), greater than or equal to 0.5% (wt/wt), greater than or equal to 1% (wt/wt), greater than or equal to 5% (wt/wt), greater than or equal to 10% (wt/wt), greater than or equal to 15% (wt/wt), greater than or equal to 20% (wt/wt). In other embodiments, the hydrophilic polymer present within the swellable hydrogel at a concentration less than or equal to 20% (wt/wt), less than or equal to 15% (wt/wt), less than or equal to 10% (wt/wt), less than or equal to 5% (wt/wt), less than or equal to 1% (wt/wt), less than or equal to 0.5% (wt/wt), less than or equal to 0.1% (wt/wt), etc. Combinations of these percentages are also possible in certain embodiments. For example, in some embodiments the hydrophilic polymer is present within the swellable hydrogel at a concentration greater than or equal to 0.1% (wt/wt) and less than or equal to 20% (wt/wt).

In some embodiments, a hydrophilic polymer within the swellable hydrogel may have a number average molecular weight (i.e., mole fraction of molecules in a polymer sample) of between 1000 and 400,000. In some embodiments, the number average molecular weight is greater than 1000, greater than 5000, greater than 10,000, greater than 50,000, greater than 100,000, greater than 200,000, greater than 300,000, greater than 400,000, etc. In other embodiments, the number average molecular weight is less than or equal to 400,000, less than or equal to 300,000, less than or equal to 200,000, less than or equal to 100,000, less than or equal to 50,000, less than or equal to 10,000, less than or equal to 5000, less than or equal to 1000, etc. Combinations of these are possible in certain embodiments. For example, in some embodiments the number average molecular weight of the hydrophilic polymer in the swellable hydrogel is greater than or equal to 1000 and less than or equal to 400,000.

In certain embodiments, a hydrophilic polymer within the swellable hydrogel may have a weight average molecular weight (i.e., the weight fraction of molecules in a polymer sample) of between 1000 and 400,000. In some embodiments, the weight average molecular weight is greater than 1000, greater than 5000, greater than 10,000, greater than 50,000, greater than 100,000, greater than 200,000, greater than 300,000, greater than 400,000, etc. In other embodiments, the weight average molecular weight is less than or equal to 400,000, less than or equal to 300,000, less than or equal to 200,000, less than or equal to 100,000, less than or equal to 50,000, less than or equal to 10,000, less than or equal to 5000, less than or equal to 1000, etc. Combinations of these are possible in certain embodiments. For example, in some embodiments the weight average molecular weight of the hydrophilic polymer in the swellable hydrogel is greater than or equal to 1000 and less than or equal to 400,000.

In some embodiments, a hydrophilic polymer within the swellable hydrogel may have a polydispersity index (i.e., the ratio of the weight average molecular weight to the number average molecular weight) of between 1 and 5. In some cases, the polydispersity index is greater than or equal to 1, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2, greater than or equal to 3, greater than or equal to 4, greater than or equal to 5, etc. In other cases, the polydispersity index is less than or equal to 5, less than or equal to 4, less than or equal to 3, less than or equal to 2, less than or equal to 1.9, less than or equal to 1.8, less than or equal to 1.7, less than or equal to 1.6, less than or equal to 1.5, less than or equal to 1.4, less than or equal to 1.3, less than or equal to 1.2, less than or equal to 1.1, less than or equal to 1.0, etc. Combinations of these are possible in certain embodiments. For example, in some embodiments the polydispersity index of the hydrophilic polymer in the swellable hydrogel is greater than or equal to 1 and less than or equal to 5.

In some embodiments, a hydrophilic polymer (e.g., homopolymer, branched polymer, and/or block polymer) may be purchased through a commercial vendor (e.g., Sigma, BASF, etc.) or synthesized using any method known to those of skill in the art. For example, in some embodiments, a step-growth polymerization reaction may be used to produce the polymer or copolymers; in other cases, a chain-growth polymerization reaction (e.g., free radical polymerization, ionic polymerization, coordination polymerization, living polymerization, ring-opening polymerization, and reversible-deactivation polymerization) may be used to produce the polymers, as contemplated herein. Other synthetic routes are also possible, e.g., polycondensation and addition polymerization.

The swellable hydrogels disclosed herein may synthesized using any suitable method known to the skilled artisan. For example, in some embodiments, a step-growth polymerization reaction may be used to produce the swellable hydrogels; in other cases, a chain-growth polymerization reaction (e.g., free radical polymerization, ionic polymerization, coordination polymerization, living polymerization, ring-opening polymerization, and reversible-deactivation polymerization) may be used to produce the swellable hydrogels, as contemplated herein. Other synthetic routes are also possible, e.g., polycondensation and addition polymerization. In some embodiments, a photo-polymerization reaction is used to produce the swellable hydrogels. Those of skill in the art will understand that photo-polymerization reactions require a photoinitiator, in the presence of UV light (e.g., 365 nm) to initiate the polymerization reaction. Exemplary photo-initiators contemplated herein include water-soluble photo-initiators such as Lithium phenyl (2,4,6-trimethylbenzoyl) phosphinate, Ethyl (2,4,6-trimethylbenzoyl)phenylphosphinate-L.

In some embodiments, the photo-polymerization reactions comprise a monomer, and optionally, a crosslinking agent. In some embodiments, the monomer is a polysaccharide (e.g., Guar Gum, Arabic Gum, Xanthan Gum, agar, pectin, starch, carrageenan, and alginate). In some embodiments, the crosslinking agent is a hydrophilic polymer (e.g., polyethylene glycol acrylate, polyethylene glycol diacrylate, polyethylene glycol methacrylate, dimethylaminoethyl methacrylate, etc.).

In some embodiments, a swellable hydrogel is in a dried state and comprises less than or equal to 0.1% water (wt/wt). In some embodiments, the swellable hydrogel comprises less than or equal to 0.1% water (wt/wt), less than or equal to 0.05% water (wt/wt), less than or equal to 0.01% water (wt/wt), less than or equal to 0.005% water (wt/wt), or less than or equal to 0.001% water (wt/wt) in a dried state.

In some embodiments, a swellable hydrogel is configured to absorb fluid (e.g., exudate) when placed into contact with a fluid. In some embodiments, the swellable hydrogel has a gravimetric swelling ratio of between 1000% and 10,000%. In some embodiments, the swellable hydrogel has a gravimetric swelling ratio of greater than or equal to 1000%, greater than or equal to 2000%, greater than or equal to 3000%, greater than or equal to 4000%, greater than or equal to 5000%, greater than or equal to 6000%, greater than or equal to 7000%, greater than or equal to 8000%, greater than or equal to 9000%, or greater than or equal to 10,000%. In some embodiments, the swellable hydrogel has a gravimetric swelling ratio of less than or equal to 10,000%, less than or equal to 9000%, less than or equal to 8000%, less than or equal to 7000%, less than or equal to 6000%, less than or equal to 5000%, less than or equal to 4000%, less than or equal to 3000%, less than or equal to 2000%, or less than or equal to 1000%. Combinations are also possible in some embodiments. For example, in some embodiments, the swellable hydrogel has a gravimetric swelling ratio of greater than or equal to 1000% and less than or equal to 10,000%.

In some embodiments, a (super) absorbent material, as disclosed herein, may be any (super) absorbent material known to the skilled artisan. In some cases, the (super) absorbent material may be a commercially available product (e.g., Gelfoam®). In other cases, the (super) absorbent material may be synthesized by the skilled artisan using known materials and methods (e.g., polyacrylate gels/sponges such as those used in diapers). Other exemplary embodiments, included but are not limited to, Gelfoam®, nitrocellulose gauze, alginate, polyurethane, silicone foam, cellulose, and fibronectin.

In some embodiments, a (super) absorbent material has a gravimetric swelling ratio of between 1000% and 10,000%. In some embodiments, the (super) absorbent material has a gravimetric swelling ratio of greater than or equal to 1000%, greater than or equal to 2000%, greater than or equal to 3000%, greater than or equal to 4000%, greater than or equal to 5000%, greater than or equal to 6000%, greater than or equal to 7000%, greater than or equal to 8000%, greater than or equal to 9000%, or greater than or equal to 10,000%. In some embodiments, the (super) absorbent material has a gravimetric swelling ratio of less than or equal to 10,000%, less than or equal to 9000%, less than or equal to 8000%, less than or equal to 7000%, less than or equal to 6000%, less than or equal to 5000%, less than or equal to 4000%, less than or equal to 3000%, less than or equal to 2000%, or less than or equal to 1000%. Combinations are also possible in some embodiments. For example, in some embodiments, the (super) absorbent material has a gravimetric swelling ratio of greater than or equal to 1000% and less than or equal to 10,000%.

In some embodiments, a swellable hydrogel and/or (super) absorbent material is ionically charged (e.g., comprises a net positive or net negative charge). The skilled artisan will understand that such materials may absorb differing volumes of fluid depending on the pH, charge, and ionic strength of said fluid. Accordingly, in some embodiments, the swellable hydrogel and/or (super) absorbent material has a gravimetric swelling ratio of greater than or equal to 1000% (wt/wt) after 1 hour of exposure to a phosphate buffer solution with solution with a phosphate buffer concentration of 10 mM, 2.7 mM potassium chloride, 137 mM sodium chloride, and 1.76 mM potassium phosphate. pH 7.4±0.2 (25° C.). at pH 5.5. In some embodiments, the swellable hydrogel and/or (super) absorbent material has a gravimetric swelling ratio of greater than or equal to 1000% (wt/wt) after 1 hour of exposure to a phosphate buffer solution at pH 7.5. In some embodiments, the swellable hydrogel and/or (super) absorbent material has a gravimetric swelling ratio of greater than or equal to 1000% (wt/wt) after 1 hour of exposure to a phosphate buffer solution at pH 8.5.

According to some embodiments, a graft, as disclosed herein, comprises a therapeutic agent. In some embodiments, the graft comprises a swellable hydrogel and/or a (super) absorbent material. In some embodiments, the therapeutic agent is entrapped within the swellable hydrogel and/or (super) absorbent material, as disclosed herein.

Therapeutic agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. In certain embodiments, the therapeutic agent is present in the shaped graft at a concentration such that, upon release from the shaped graft, the therapeutic agent elicits a therapeutic response.

In some embodiments, a swellable hydrogel comprises a therapeutic agent. In some embodiments, a (super) absorbent material comprises the therapeutic agent. In some embodiments, the therapeutic agent is present in the swellable hydrogel (e.g., second layer) and the (super) absorbent material (e.g., third layer). In some embodiments, a graft comprises two or more therapeutic agents (e.g., a first therapeutic agent and a second therapeutic agent different than the first therapeutic agent).

Therapeutic agents can be loaded into various layers of graft, as disclosed herein, via standard methods including, but not limited to, powder mixing, direct addition, solvent loading, melt loading, physical blending, supercritical carbon dioxide assisted, and conjugation reactions such as ester linkages and amide linkages. Release of therapeutic, diagnostic agents can then be accomplished through methods including, but not limited to, dissolution of the components/materials, degradation of the components/materials, swelling of the components/materials, diffusion of an agent, hydrolysis, and chemical or enzymatic cleavage of conjugating bonds. In some embodiments, the therapeutic agent is covalently bound to the components/materials (e.g., and is released as the components/materials degrade).

In some embodiments, the therapeutic agent is embedded within one or more layers of a graft, as disclosed herein. In some embodiments, the therapeutic agent is associated with the graft (or one or more components/regions/materials therein) via formation of a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups.

In some embodiments, a therapeutic agent is an antimicrobial agent (e.g., silver nano particles, gold nano particles, Zinc peroxide, thyme oil, cinnamon oil, clove oil, lavender oil, eucalyptus oil, cardamom oil, manuka honey, ampicillin, erythromycin, Ciprofloxacin, methicillin, Daptomycin, Gentamicin, Poly hexamethylene, octenidine dihydrochloride and Biguanide (PHMB)) and other agents known to those skilled in the art.

In some embodiments, the therapeutic agent is in the form of a layer of material that binds and removes bacteria from the wound bed and peri-wound area (e.g., sorbact).

In some embodiments, a therapeutic agent is an antifungal agent (e.g., fluconazole, itraconazole, ketoconazole, miconazole, nystatin, terbinafine, voriconazole), or the like, known to those skilled in the art.

Other aspects of the present disclosure relate to methods for assembling a graft as disclosed herein. In some embodiments, two adjacent layers (e.g., a second layer disposed on a first layer, a third layer disposed on a second layer, or a fourth layer disposed on a third layer, etc.), may be joined to each other using a bio-glue, such as a cyanoacrylate. In some embodiments, two adjacent layers may be joined using a cyanoacrylate dot.

Other methods of joining adjacent layers are also possible. For example, in some embodiments, a second layer comprising a swellable hydrogel and a third layer comprising a (super) absorbent material may be joined by partially crosslinking the swellable hydrogel around the (super) absorbent material (e.g., cellulose, cotton, gel foam, nitrocellulose gauze, alginate, polyurethane, silicone foam, and nitrocellulose, and fibronectin). This may be accomplished, for example, by infusing a polymerizable liquid solution (e.g., hydrogel forming liquid solution) into at least part of the bulk (e.g., within the interstitial space) of the (super) absorbent material. Polymerization of the liquid solution creates an swellable hydrogel network that interpenetrates the porous network of the super (absorbent) material, thus physically crosslinking the two layers together.

In some embodiments, the methods comprise cutting a graft to conform to the geometry of the wound (e.g., using a stencil). In some embodiments, cutting is accomplished using a razor blade. In some embodiments, cutting is accomplished using a laser cutter. In some embodiments, cutting is accomplished using a waterjet cutter. In some embodiments, cutting a plasma cutter. Any of the aforementioned cutting methods may be performed manually according to one set of embodiments. However, in another set of embodiments, the cutting process is automated using, for example, a computer. In some embodiments, the cutting process is performed by a computer, or alternatively, a machine controlled by a computer.

In some embodiments, the methods comprise cleaning the wound to ensure the wound is free from active infection (e.g., bacterial infection) prior to adding the graft. In some embodiments, cleaning the wound does not comprise using betadine. In some embodiments, the methods comprise debridement of the wound (e.g., surgical debridement). In some embodiments, the methods comprise opening an outer peel pouch to expose the sterile shaped graft. In some embodiments, the methods comprise placing the sterile shaped graft directly onto the granular wound bed. Additionally, the methods comprise anchoring the shaped graft to the subject, according to one set of embodiments. Any method of fixation known to the skilled artisan may be used to anchor the shaped graft (e.g., use of an adhesive layer).

In some embodiments, the method comprises evaluating the wound and reapplying a new sterile shaped graft as needed until the wound heals. Any period of time may pass before the wound is evaluated and a new shaped graft applied. In some embodiments, a new shaped graft is applied every day, every other day, every third day, every fourth day, and every fifth day. In some embodiments the new shaped graft is applied at least once monthly, at least twice monthly, at least three times monthly, at least four times monthly, and at least five times monthly, at least 6 times monthly, at least 7 times monthly, at least 8 times monthly, at least 9 times monthly, and at least 10 times monthly.

### EXAMPLES

### 1. Fabrication of Antimicrobial Silver Nanoparticle-Loaded PEGDA-Guar Gum Hydrogel.

Antimicrobial silver nanoparticle-loaded PEGDA-Guar Gum hydrogels were prepared as follows: 0.5625 grams of PEGDA 20k was added to 2.25 mL of dH2O in a falcon tube and vertexing until full dissolution occurred (the solution was clear with no undissolved PEGDA 20k solid) to yield a 30 wt % PEGDA 20k solution. 0.75 mL (15 wt %) of 30 wt % PEGDA 20k solution was then added into 3 separate 2 mL Eppendorf tubes. 0.15 grams of guar gum was added to each vial for a final concentration of 10% (See Table 1). To each vial was then added 7.5 mg (0.5 wt %) of LithiumPhenyl-2,4,6-trimethylbenzoylphosphinate (LAP) and the vials vortexed until the photoinitiator was fully dissolved. Each solution was then pipetted into a separate frame adhered to a Teflon surface modified glass microscope slide and the solutions irradiated at 365 nm for 5 minutes from about 10 cm away. After polymerization, the gels were oven dried at 80 degrees Celsius for 60 minutes. After drying, the gels were peeled from the frames and 5 mm circular membrane disks were prepared using a biopsy punch. It was noted that deformation of the gels occurred during drying.

TABLE 1
PEGDA-GG Hydrogel Composition
by Weight (per 1.5 mL sample)

| | | Total | Composition |
|---|---|---|---|
| | Material | Weight | of Gel Batch |
| Material | MW (Da) | Added (g) | (wt %) |
| Polyethylene(glycol) | 20,000 | 0.225 | 15 |
| Diacrylate (PEGDA) | | | |
| Guar gum (GG) | 1-2 × 10\hat{ }6 | 0.15 | 10 |
| Lithium phenyl-2,4,6- | 294.210 | 0.0075 | 0.5 |
| trimethylbenzoylphosphinate | | | |
| (LAP) | | | |
| Deionized Water (dH2O) | 18.015 | 1.1175 | 74.5 |
| | Total | 1.5 | 100 |

### 2. Swelling Studies of PEGDA-Guar Gum Hydrogels at Varying pH.

Stock PBS solutions of pH 5.5, 7.5, and 8.5 were prepared using methods known in the art. PEGDA-Guar Gum hydrogels were fabricated according to Table 1. These samples were initially weighed (t=0), then placed individually in numbered wells. To each well, At the times t=1, 2, 3, 4, 7, 24, 48, and 72 hours, the samples were removed from the respective PBS solution, delicately scraped/wiped of excess PBS as much as possible, and reweighed to assess how much solution swelled into the hydrogel network (was absorbed) during the elapsed time. After all reweights were complete, the data was analyzed and swelling curves were produced. FIG. 9 shows a plot of the resulting data. After one hour, the gels swelled in PBS pH 8.5 were softest, while the gels swelled in PBS pH 5.5 were most rigid.

### 2. Adhesion of Dried Hydrogel Layer and Secondar Dressing Layer Using Cyanoacrylate Glue.

FIGS. 11A and 11B show exemplary grafts comprising a second layer comprising a swellable hydrogel and a third layer comprising a (super) absorbent material, wherein the layers were attached using cyanoacrylate glue. In FIG. 11A, the second layer and third layer were attached by applying the cyanoacrylate glue to the outer edge of the third layer. In FIG. 11B, the second layer and third layer were attached by applying a cyanoacrylate dot in the center of the third layer. As shown in FIG. 11B, the cyanoacrylate dots allowed the hydrogel to swell freely and did not lead to warping as observed in FIG. 11A. In general, swelling was about 12.5 times the grafts dry weight. In some embodiments, the swellable hydrogel further comprises silver calcium alginate. In some embodiments, the swellable hydrogel further comprises silver polyurethane. In some embodiments, the (super) absorbent material further comprises silver calcium alginate. In some embodiments, the super) absorbent material further comprises silver polyurethane.

### 3. Adhesion of Dried Hydrogel Layer and Secondary Dressing Layer Via Crosslinking.

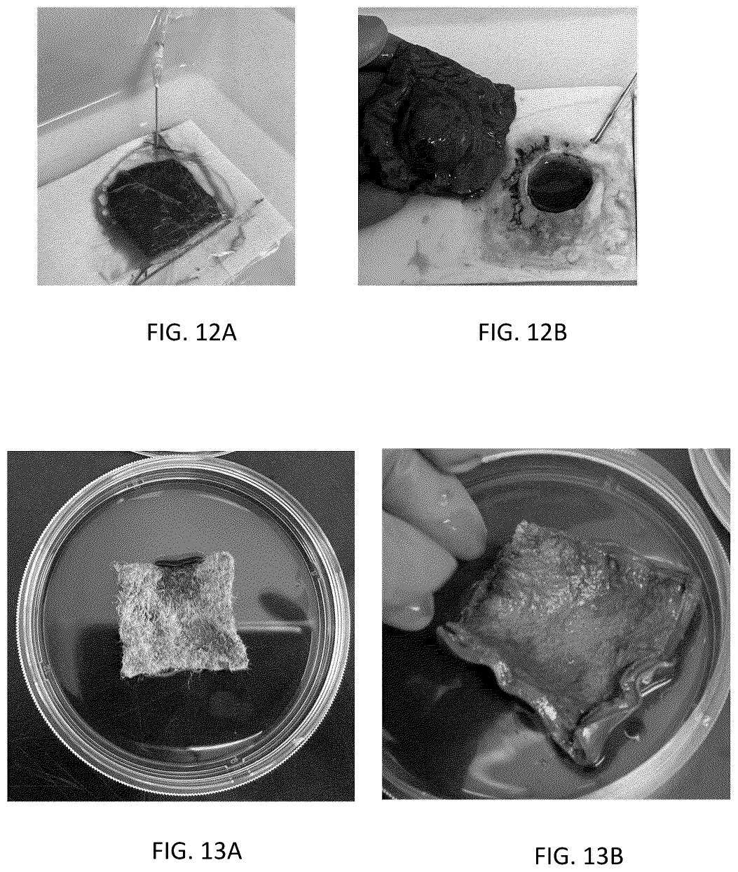

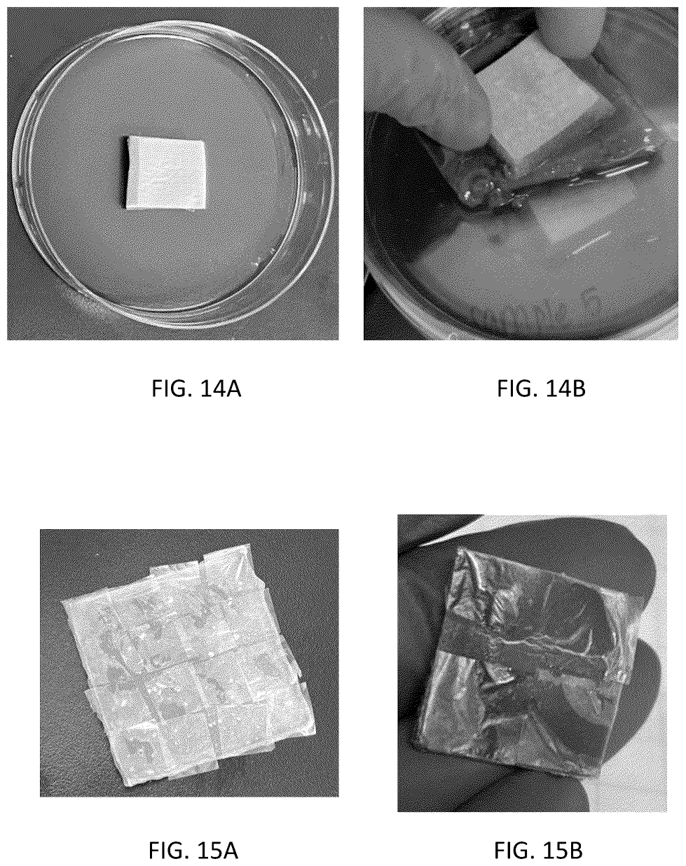

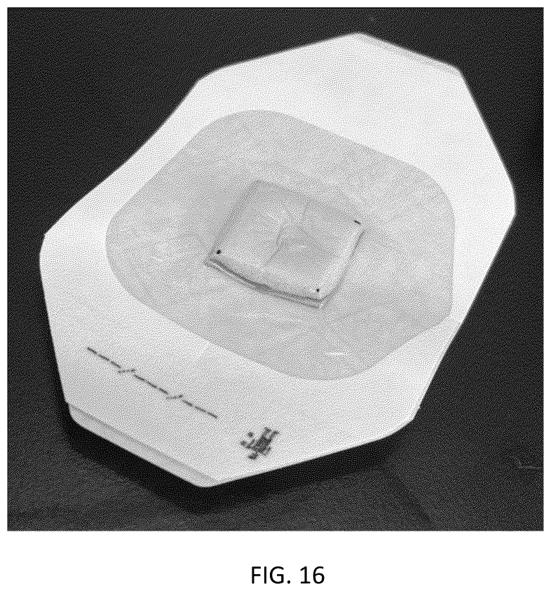

FIGS. 12A and 12B show exemplary grafts comprising a second layer comprising a swellable hydrogel and a third layer comprising a (super) absorbent material, wherein the swellable hydrogel is crosslinked, at least partially, around the (super) absorbent material. This is accomplished by infusing a swellable hydrogel liquid photopolymerizable precursor solution directly into the (super) absorbent material and subsequently photopolymerizing the swellable hydrogel, within at least a part, of the (super) absorbent material. In general, swelling was about 10 times the grafts dry weight. In some embodiments, the swellable hydrogel further comprises silver calcium alginate. In some embodiments, the swellable hydrogel further comprises silver polyurethane. In some embodiments, the (super) absorbent material further comprises silver calcium alginate. In some embodiments, the super) absorbent material further comprises silver polyurethane.

4. Division of First Layer into a Plurality of Segments and Attachment to a Second Layer.

FIG. 15A shows an exemplary first layer comprising amnion, chorion, Wharton's jelly, cord, or a combination thereof, divided into a plurality of segments. FIG. 15B shows an exemplary graft wherein each segment is disposed directly onto a first side of a second layer comprising a swellable hydrogel.

### EQUIVALENTS AND SCOPE

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. Section 2111.03.

What is claimed is:

1. A graft having exudate control for treating a wound of a subject, the graft comprising a layer comprising:
a first portion comprising amnion, chorion, Wharton's Jelly, cord or a combination thereof; and
a second portion adjacent the first portion, comprising a swellable hydrogel,
wherein the swellable hydrogel comprises a hydrophilic polymer and a polysaccharide.

2. The graft as in claim 1, wherein the first portion is in the form of a membrane.

3. The graft as in claim 1, wherein the first portion is in the form of suspended particles of amnion, chorion, Wharton's jelly, or cord that is 3D Printed into a layer.

4. The graft as in claim 1, wherein the first portion is comprised of fibers of amnion, chorion, Wharton's Jelly, cord or combination of; that have been spun into a matt.

5. The graft as in claim 1, wherein the first portion is comprised of fibers of amnion, chorion, Wharton's Jelly, cord or combination of; that have been bundled and turned into a mesh

6. The graft as in claim 1, wherein the first portion is comprised of amnion, chorion, Wharton's Jelly, cord or combination of; that have been lyophilized into a foam.

7. The graft as in claim 1, wherein the second portion is disposed within a hole of the first portion.

8. The graft as in claim 1, wherein the first portion comprises two or more graft layers, each of the graft layers being the same or different and comprising amnion, chorion, Wharton's Jelly, cord or a combination thereof.

9. The graft as in claim 1, further comprising a second layer adjacent the first layer comprising a hydrophilic superabsorbent material (e.g., gel foam, nitrocellulose gauze, alginate, polyurethane, silicone foam, cellulose, and nitrocellulose. fibronectin).

10. The graft as in claim 1, further comprising an adhesive layer adjacent the first second layer for securing the graft to the subject.

11. The graft as in claim 1, wherein the hydrophilic polymer is selected from the group consisting of polyethylene glycol, polyethylene glycol acrylate, polyethylene glycol diacrylate, polyethylene glycol methacrylate, polyacrylamide, polyvinyl alcohol, polyoxaester, Pluronic, dimethylaminoethyl methacrylate, poly(2-hydroxyethyl methacrylate), hyaluronic acid, poly (N-isopropylacrylamide), poly ethylene oxide, poly oxaesters, chitosan, and dextran.

12. The graft as in claim 1, wherein the polysaccharide is selected from the group consisting of guar gum, Arabic gum, gellan gum, Xanthan Gum, agar, pectin, starch, carrageenan, and alginate.

13. (canceled)

14. The graft as in claim 1, further comprising an antimicrobial agent (e.g., silver nano particles, gold nano particles, Zinc peroxide, thyme oil, cinnamon oil, clove oil, lavender oil, eucalyptus oil, cardamom oil, manuka honey, ampicillin, erythromycin, Ciprofloxacin, methicillin, Daptomycin, Gentamicin, Poly hexamethylene, Octenidine Dihydrochloride, and Biguanide (PHMB)) or a layer of antimicrobial material that binds and removes bacteria.

15. (canceled)

16. The graft as in claim 1, further compromising an antifungal agent (e.g., fluconazole, itraconazole, ketoconazole, miconazole, nystatin, terbinafine, voriconazole).

17. The graft as in claim 1, wherein the graft exhibits greater than or equal to 1000% gravimetric swelling after 1 hour in pH 5.5 phosphate buffer solution, greater than or equal to 1000% gravimetric swelling after 1 hour in pH 7.5 phosphate buffer solution, and/or greater than or equal to 1000% gravimetric swelling after 1 hour in pH 8.5 phosphate buffer solution.

18. The graft as in claim 1, wherein the graft has a shape corresponding to the wound of the subject.

19. A graft having exudate control for treating a wound of a subject, the graft comprising:
a first layer comprising amnion, chorion, or a combination thereof; and
a second layer, disposed on the first layer, comprising a swellable hydrogel,
wherein the swellable hydrogel comprises a hydrophilic polymer and a polysaccharide.

20. The graft of claim 19, wherein the first layer is divided into a plurality of segments comprising amnion, chorion, Whaton's Jelly, cord, or a combination thereof.

21. The graft of claim 19, wherein the first layer comprises a single hole or a plurality of holes.

22-24. (canceled)

25. The graft of claim 19, further comprising a third layer comprising an absorbent material (e.g., gel foam, nitrocellulose gauze, alginate, polyurethane, silicone foam, cellulose, and nitrocellulose. fibronectin), wherein the third layer is disposed on the second layer.

26-27. (canceled)
